(19) United States
(12) Patent Application Publication
(10) Pub. No.: US 2024/0172958 A1
(43) Pub. Date: May 30, 2024

(54) DATA FUSION FOR CONTACTLESS ESTIMATION OF RESPIRATION RATE

(71) Applicant: KONINKLIJKE PHILIPS N.V., EINDHOVEN (NL)

(72) Inventors: Ikaro Garcia Araujo da Silva, Cambridge, MA (US); Daniel Jason Schulman, Cambridge, MA (US); Sara Mariani, Cambridge, MA (US)

(21) Appl. No.: 18/510,793

(22) Filed: Nov. 16, 2023

Related U.S. Application Data
(60) Provisional application No. 63/428,566, filed on Nov. 29, 2022.

(51) Int. Cl. A61B 5/08; A61B 5/00; A61B 5/113

(52) U.S. Cl. CPC A61B 5/0816; A61B 5/0077; A61B 5/0803; A61B 5/113; A61B 5/7221; A61B 5/7271; A61B 5/742; A61B 2562/0204

(57) ABSTRACT

A system for estimating respiration rate is provided. The system includes an audio sensor, a displacement sensor, and a processing unit. The audio sensor, such as a parabolic microphone, produces an audio recording signal. The displacement sensor, such as a camera or laser rangefinder, produces a chest movement signal. The processing unit receives the audio recording signal and the chest movement signal. The processing unit computes an estimated audio respiration rate based on the audio recording signal and an estimated displacement respiration rate based on the chest movement signal. The processing unit computes an audio signal quality index for the audio recording signal and a displacement signal quality index for the chest movement signal. The processing unit determines a fused estimated respiration rate by fusing the estimated audio respiration rate with the estimated displacement respiration rate. The fusing is based on the audio and displacement signal quality indexes.

### CROSS-REFERENCE TO RELATED APPLICATIONS

This application claims priority to U.S. Provisional Patent Application Ser. No. 63/428,566, filed on Nov. 29, 2022, and titled “Data Fusion for Contactless Estimation of Respiration,” which application is herein incorporated by reference in its entirety.

### FIELD OF THE DISCLOSURE

The disclosed subject matter generally pertains to long-distance contactless estimation of respiration rate.

### BACKGROUND

Changes in respiratory rate are meaningful indicators of inflammation and infection. Long distance contactless monitoring of respiration rates for infection surveillance is desirable in scenarios where wearable sensors or short-distance measurement (e.g., in a fixed-location kiosk setting) are impractical, including battlefield, disaster area, emergency room, and staff monitoring scenarios.

### SUMMARY OF THE DISCLOSURE

The present disclosure is directed to a system for estimating respiration rate from the fusion of different modalities of respiration rate estimation. The different modalities include audio recording signals generated by one or more microphones or other audio sensors and chest movement signals generated by one or more cameras, laser rangefinders, or other displacement sensors. The different modalities are fused together based on signal quality indexes associated with each modality, such as via weighted averaging.

Generally, in one aspect, a system for estimating respiration rate is provided. The system includes an audio sensor. The audio sensor is configured to produce an audio recording signal.

The system further includes a displacement sensor. The displacement sensor is configured to produce a chest movement signal.

The system further includes a processing unit. The processing unit is configured to receive the audio recording signal and the chest movement signal.

The processing unit is further configured to compute an estimated audio respiration rate and an estimated displacement respiration rate. The estimated audio respiration rate is computed based on the audio recording signal and the estimated displacement respiration rate is computed based on the chest movement signal.

The processing unit is further configured to compute an audio signal quality index for the audio recording signal and a displacement signal quality index for the chest movement signal.

The processing unit is further configured to compute a fused estimated respiration rate by fusing the estimated audio respiration rate with the estimated displacement respiration rate. The fusing is based on the audio signal quality index and the displacement signal quality index.

The processing unit is further configured to output the fused estimated respiration rate.

According to an example, the audio signal quality index and/or the displacement signal quality index is computed in epochs of 30 seconds.

According to an example, the fusing of the estimated audio respiration rate and the estimated displacement respiration rate is done by using weighted averaging of the audio signal quality index and the displacement signal quality index.

According to an example, the estimated audio respiration rate is determined through spectrogram analysis of the audio recording signal.

According to an example, the estimated displacement respiration rate is calculated based on a power spectrum density of the chest movement signal.

According to an example, the audio signal quality index is calculated based on a respiration rate frequency range and a total frequency range of the audio recording signal.

According to an example, the displacement signal quality index is calculated based on a power spectrum density of the chest movement signal.

According to an example, the system further includes a display unit. The display unit is configured to display the fused estimated respiration rate.

According to an example, the audio sensor is a parabolic microphone.

According to an example, the displacement sensor is a camera.

According to an example, the displacement sensor is a laser rangefinder.

Generally, in another aspect, a method for estimating respiration rate is provided. The method includes receiving, via a processing unit, an audio recording signal and a chest movement signal.

The method further includes computing, via the processing unit, an estimated audio respiration rate based on the audio recording signal and an estimated displacement respiration rate based on the chest movement signal.

The method further includes computing, via the processing unit, an audio signal quality index for the audio recording signal and a displacement signal quality index for the chest movement signal.

The method further includes computing, via the processing unit, a fused estimated respiration rate by fusing the estimated audio respiration rate with the estimated displacement respiration rate based on the audio signal quality index and the displacement signal quality index.

The method further includes outputting, via the processing unit, the fused estimated respiration rate.

According to an example, the method further includes displaying, via a display unit, the fused estimated respiration rate.

According to an example, the method further includes producing the audio recording signal via parabolic microphone.

According to an example, the method further includes producing the chest movement signal via a camera or a laser rangefinder.

In various implementations, a processor or controller may be associated with one or more storage media (generically referred to herein as “memory,” e.g., volatile and non-volatile computer memory such as RAM, PROM, EPROM, EEPROM, floppy disks, compact disks, optical disks, magnetic tape, SSD, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is an illustration of a system for estimating respiration rate, in accordance with an example.

FIG. 2A is a block diagram of a system for estimating respiration rate, in accordance with an example.

FIG. 2B is a block diagram of a variation of the system for estimating respiration rate shown in FIG. 2A, in accordance with an example.

FIG. 2C is a block diagram of a variation of the systems for estimating respiration rate shown in FIGS. 2A and 2B, in accordance with an example.

FIG. 3 is a time domain plot of data captured by a camera, in accordance with an example.

FIG. 4 is a power spectrum density plot of data captured by a camera, in accordance with an example.

FIG. 5 is a schematic diagram of a controller of a system for estimating respiration rate, in accordance with an example.

FIG. 6 is a flow chart of a method for estimating respiration rate, in accordance with an example.

### DETAILED DESCRIPTION OF EMBODIMENTS

The present disclosure is directed to a system for estimating respiration rate from the fusion of different modalities of respiration rate estimation. The different modalities include audio recording signals generated by one or more microphones or other audio sensors and chest movement signals generated by one or more cameras, laser rangefinders, or other displacement sensors. The different modalities are fused together based on signal quality indexes associated with each modality, such as via weighted averaging.

Efficient algorithms have been developed for the early prediction of infection based on laboratory values and vital signs. Specific biomarkers have been identified as most predictive of infection. Respiratory rate is one of the top predictors of infection.

This disclosure focuses on allowing monitoring of respiratory rate without the burden of wearable devices. This approach is particularly suited for situations in which it is inconvenient for the subject to wear a wristband, patch, or chest strap, like in military applications where sensors can be a burden to operations. In some examples, the monitored respiratory rate may then be used as part of an infection prediction.

This disclosure provides a data fusion algorithm that integrates respiration rates derived from audio recordings using parabolic microphones (such as parabolic microphones manufactured by Wildtronics), chest movements using cameras (such as camera manufactured by Sony or Nikon), and/or laser rangefinders (such as rangefinders manufactured by Leica) to estimate respiration rate accurately and robustly.

In particular, the disclosure describes extracting respiration signals from remote devices and integrating the respiration signals with using a custom signal quality index for each measurement signal. The estimated respiration signals can be then used as part of a broader service to provide individual estimates of infection risks for users.

The disclosure describes a detailed method to (1) estimate the signal quality index (SQI) for respiration rate estimation for all three of the modalities (audio microphone, camera, laser rangefinder), and (2) using the SQI, fuse or integrate the respiration rate across all three modalities such that final derived respiration rate is more robust to noise and data missingness, and is also more accurate than individual estimates. In particular, the method solves the problem of how to integrate multiple remote measurements of respiration rate so that the final fused estimate is more accurate and robust.

Table 1 below shows preliminary results from human recordings on both indoor and outdoor settings demonstrating that data fusion yields the best results at 20 m (Nikon camera and Wildtronics microphone data fusion) and 45 m (Sony camera and Wildtronics microphone data fusion). In particular, Table 1 shows a median absolute error value for individual camera, rangefinder, and microphone modalities, along with the fusion of different combinations of camera, rangefinder, and microphone data. Lower median absolute error values correspond to more accurate remote monitoring estimates. The median absolute error value is calculated by comparing respiration rate estimated via remote monitoring according to the aforementioned modalities to a ground truth respiration rate determined from a wearable device. As can be seen in Table 1, the disclosed fusion of data modalities makes the estimated respiration rate more robust to extreme values (see LEICA individual estimates compared to fused estimates).

TABLE 1

| | Median Absolute Error | | |
|---|---|---|---|
| | (Target: Human) | | |
| | | | 100 m |
| Sensor | 20 m | 45 m | (outdoors) |
| Sony DSCRX10 camera | 1.01 | 1.39 | 0.86 |
| Nikon Coolpix P1000 camera | 0.76 | 3.69 | 2.17 |
| Leica D810 rangefinder | 5.74 | 8.25 | 13.76 |
| Wildtronics microphone | 3.74 | 3.49 | 7.70 |
| Sony camera + Leica rangefinder (SQI fusion) | 1.16 | 2.04 | 1.75 |
| Nikon camera + Leica rangefinder (SQI fusion) | 0.89 | 4.02 | 2.17 |
| Sony camera + Wildtronics microphone (SQI | 1.32 | 1.34 | 0.93 |
| fusion) | | | |
| Nikon camera + Wildtronics microphone (SQI | 0.69 | 3.35 | 2.17 |
| fusion) | | | |

In order to implement a system according to this disclosure, the following elements are needed: (1) a commercial, off-the-shelf parabolic microphone, a camera, and a rangefinder; (2) a data ingestion pipeline for all the recording devices from item 1; (3) a software module, described in this disclosure, that calculates the estimated respiration rate and the SQI for each measurement and device (in epochs of 30 seconds); and (4) a software module, described in this disclosure, that fuses all estimates from item 3 based on the SQIs for a given epoch and reports the fused estimated respiration rate.

Estimation of respiration rate in breaths per minute (BPM) is performed from the measurement of each device according to the following steps. For audio recording from the parabolic microphone, the estimated respiration rate is determined through spectrogram analysis of the audio recording signal. The audio SQI is then calculated by dividing the spectrogram frequency range used in respiration rate estimation by the total frequency range of the spectrogram. It is assumed that good quality signals will have a narrow spectrum, and bad recordings or measurements with noise will have a broad and flatter spectrum.

For either camera or laser rangefinder recordings, the respiration rate is estimated based on the power spectrum density (PSD) of the displacement signal. The signal quality for the PSD is then given by: SQI=(1−H(x)²) where H(x)=−Σᵢ₌₁ⁿP(xᵢ)log P(xᵢ). In these equations, P(xᵢ) is the PSD at a frequency xᵢ. Thus, H(x) is a measure of the bandwidth of the PSD of the signal generated by the camera or laser rangefinder. As with audio DQI, tt is assumed that good quality signals will have a narrow spectrum, and bad recordings or measurements with noise will have a broad and flatter spectrum.

Data fusion at the given epoch and across all devices is then performed by using the estimated respiration rates in BPM and SQI for each device i using weighted averaging:

$$Fused BPM = \sum {w}_{i} * B P {M}_{i} , where {w}_{i} = \frac{S Q {I}_{i}}{\sum SQI}$$

As discussed previously, aspects of this disclosure may be applied in a number of ways. For instance, aspects may be used to monitor the respiration rate of warfighters at potential risk of infection, which is important to (1) ensure their optimal performance in military operations and (2) prevent spread of pathogens in close-contact living. Additionally, aspects may be used to monitor the respiration rate of high-risk people in community living, e.g., elderly patients in a retirement home, or people in constrained quarters such as inmates, ship crew, and/or medical staff. Other applications of long-distance monitoring of respiration rate are possible.

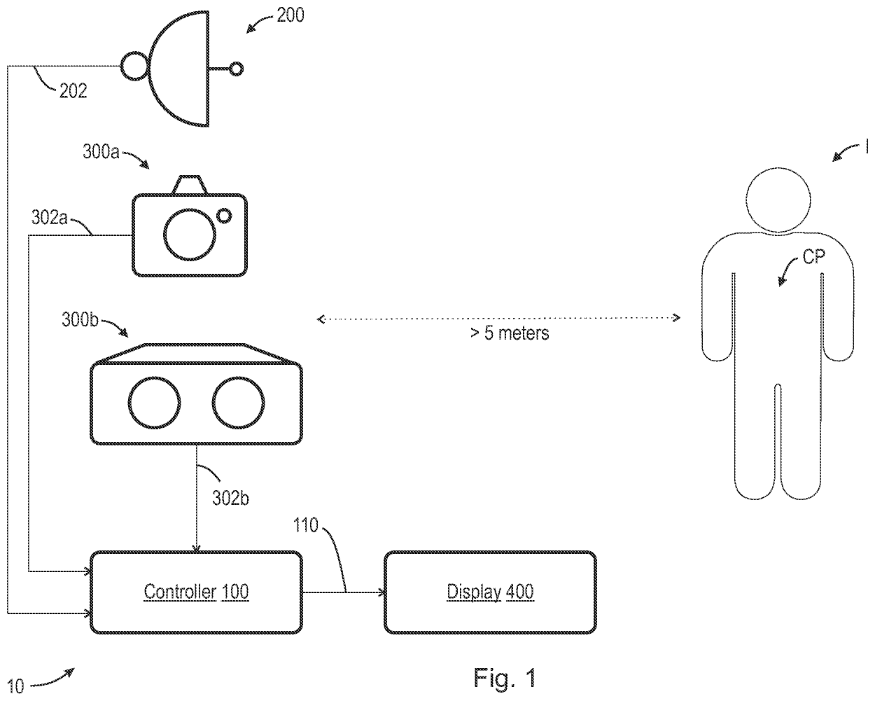

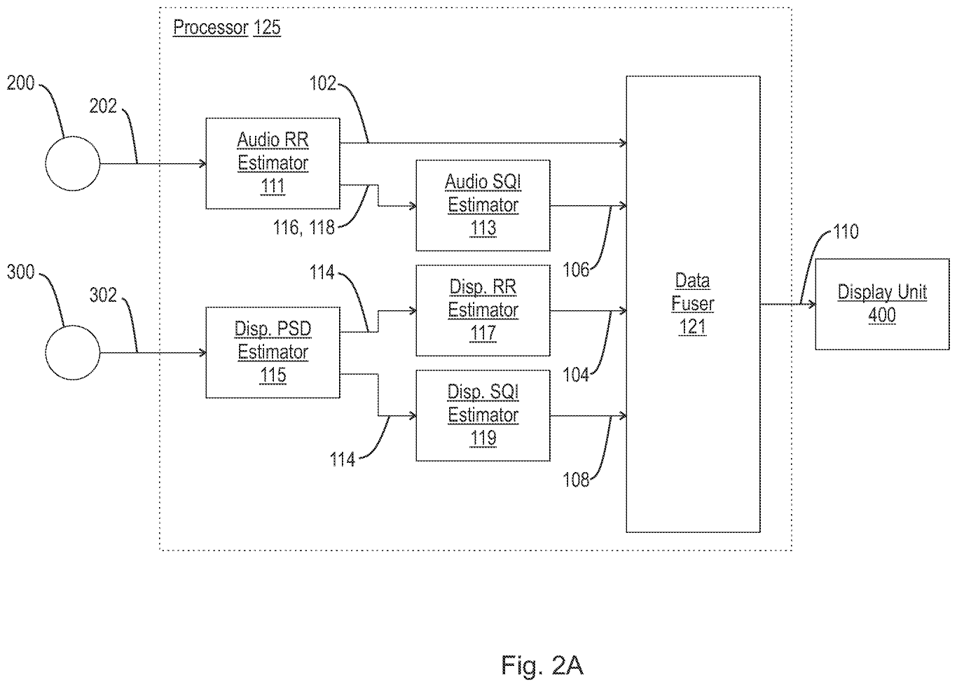

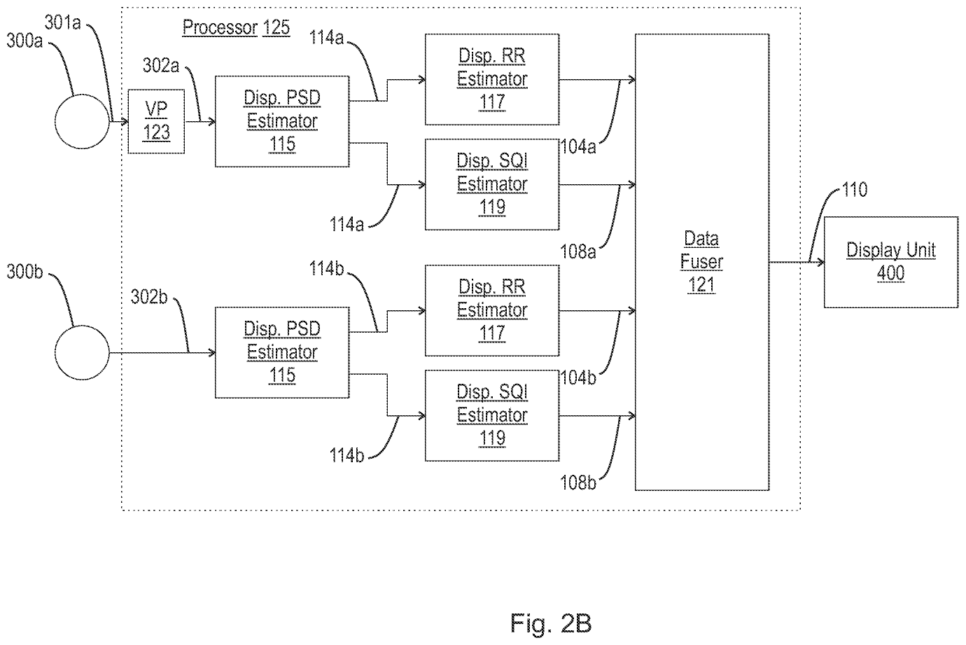

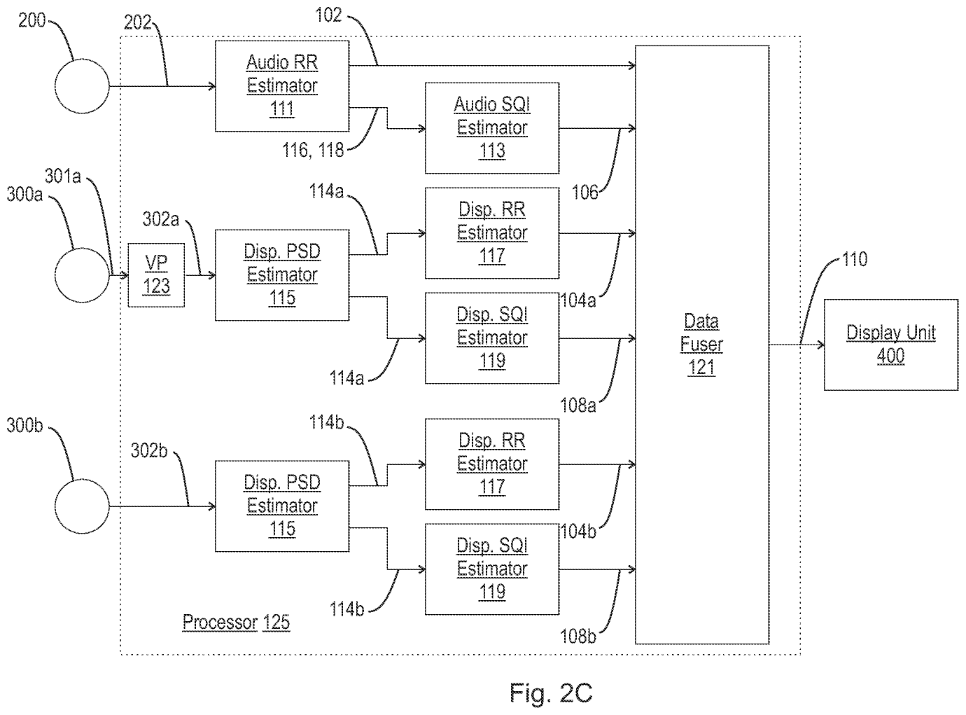

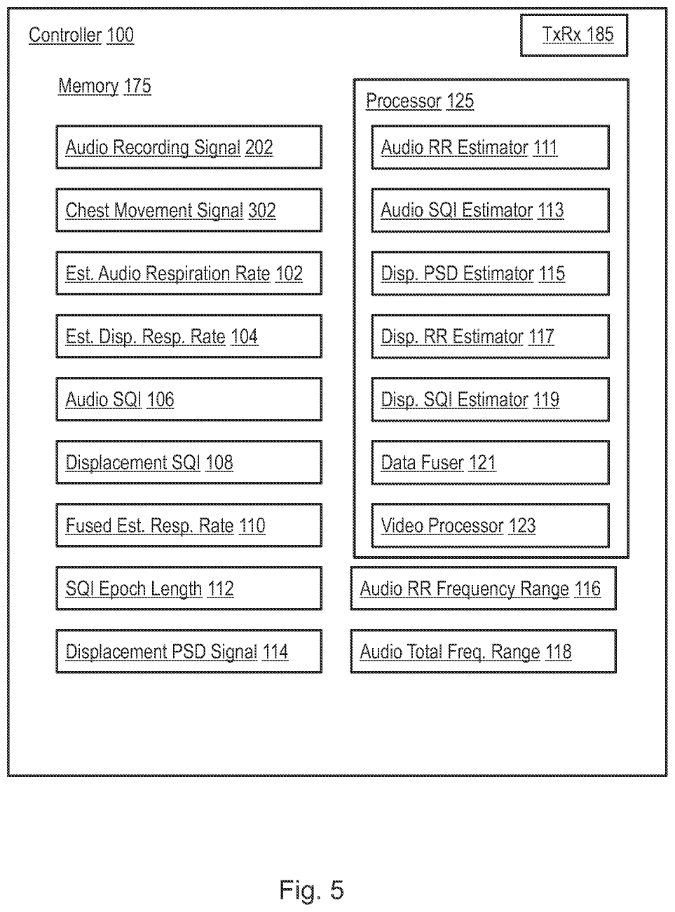

Turning now to the figures, FIG. 1 illustrates a non-limiting example of a system 10 for estimating respiration rate. As shown in FIG. 1, the system 10 generally includes a controller 100, an audio sensor 200, one or more displacement sensors 300a, 300b, and a display 400. As will be shown in more detail in FIGS. 2 and 5, the controller 100 includes a processor 125, a memory 175, and a transceiver 185. In some examples, the processor 125 may be embodied as a plurality of processing units. Similarly, the memory 175 may be embodied as a plurality of memory storage units. The transceiver 185 may be used to enable wireless communication between the controller 100 and the other aspects of the system 10, such as the audio sensor 200, the displacement sensors 300a, 300b, and/or the display 400.

The example system of FIG. 1 further includes an audio sensor 200 embodied as a parabolic microphone. Generally, the audio sensor 200 may be any device capable of capturing audio corresponding to an individual I arranged at a long-distance from the audio sensor. In some examples, the distance between the audio sensor 200 and the individual I is at least five meters. In some examples, the audio sensor 200 is a single parabolic microphone. In other examples, the audio sensor 200 may be embodied as multiple microphones, including microphones of other varieties other than parabolic microphones.

The example system 10 of FIG. 1 further includes a first displacement sensor 300a embodied as a camera and a second displacement sensor 300b embodied as a laser rangefinder. Generally, the displacement sensors 300a, 300b may be any device capable of visually capturing data reflective of chest position CP of an individual I arranged at a long-distance from the sensors 300a, 300b. In some examples, the distance between the displacement sensors 300a, 300b and the individual I is at least five meters. In some examples, the system 10 only includes a single displacement sensor 300. In other examples, the system 10 may include more than two displacement sensors 300. In even further examples, the system 10 may include two or more displacement sensors 300 of the same type, such as two or more cameras or laser rangefinders. In some examples, the camera 300a may be an RGB camera.

The audio sensor 200 is configured to transmit, via wired or wireless connection, an audio recording signal 202 to the controller 100. The first displacement sensor 300a is configured to transmit, via wired or wireless connection, a first chest movement signal 302a to the controller 100. Similarly, the second displacement sensor 300b is configured to transmit, via wired or wireless connection, a second chest movement signal 302b to the controller 100. The controller 100 processes the audio recording signal 202 and the first and second chest movement signals 202a, 202b to estimate a fused estimated respiration rate 110 of the individual I. In some examples, the controller 100 is arranged proximate to the audio sensor 200 and/or the displacement sensors 300a, 300b. In other examples, the controller 100 may be arranged a significant distance from the audio sensor 200 and the displacement sensors 300a, 300b, requiring data to be transferred from the sensors 200, 300a, 300b to the controller 100 via wireless transmission and/or cloud computing.

The example system 10 of FIG. 1 further includes a display 400. In some examples, the display 400 may be incorporated in the controller 100. In other examples, the display 400 could be incorporated into a discrete device, such as a smartphone or a personal computer. The display 400 is configured to receive, via wired or wireless connection, the estimated breath per minute data 116. The display 300 is then configured to show the fused estimated respiration rate 110 on a display screen, such as a touch screen.

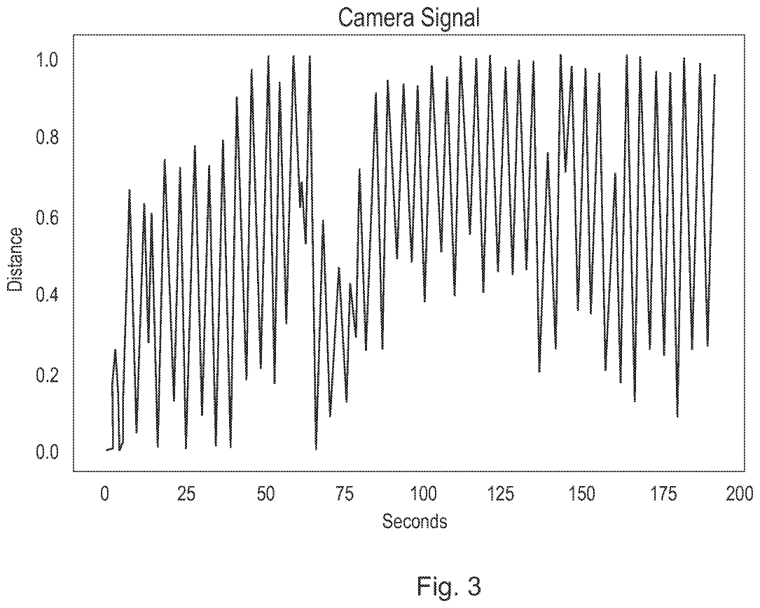

FIG. 2A illustrates a block diagram describing the data processing of an audio recording signal 202 and a chest movement signal 302 to generate a fused estimated respiration rate 110. As shown in the non-limiting example of FIG. 2A, an audio sensor 200 (such as a parabolic microphone) provides the audio recording signal 202 to the processor 125. Similarly, a displacement sensor 300 (such as a camera or a laser rangefinder) provides the chest movement signal 302 to the processor. An example of a chest movement signal 302 captured by a camera is shown in FIG. 3.

Broadly, the processor 125 is configured to process at least two different modalities of sensor data to determine a fused estimated respiration rate 110. In the non-limiting example of FIG. 2A, the modalities are audio data provided by the audio sensor 200 and the displacement data provided by the displacement sensor 300. Thus, the processor generates an estimated respiration rate based on audio data and an estimated respiration rate based on displacement data. The two estimated respiration rates are then combined based on the quality of the signals used to derive the estimates. In some examples, the processor 125 may process data from multiple types of displacement sensors 300, including both cameras 300a and laser rangefinders 300b. For example, the processor 125 could be configured to fuse data of the multiple types of displacement sensors 302 before fusing the displacement data with the audio data.

In the example of FIG. 2A, the audio recording signal 202 is provided to audio respiration rate estimator 111. The audio respiration rate estimator 111 generates an estimated audio respiration rate 102 rate through spectrogram analysis of the audio recording signal 202. Further, a respiration rate frequency range 116 and a total frequency range 118 of the spectrogram audio recording signal 102 is provided to an audio signal-quality-index (SQI) estimator 113 to determine an audio SQI 106. The respiration rate frequency range 116 represents the frequency range of the audio recording signal 202 used to determine the estimated audio respiration rate 102. Accordingly, the audio SQI 106 may be determined from the temporal correlation of the amplitude fluctuations of the frequencies of the spectrogram. The audio SQI 106 is determined according to a predefined SQI epoch length 112. In some examples, the predefined SQI epoch length 112 may be 30 seconds.

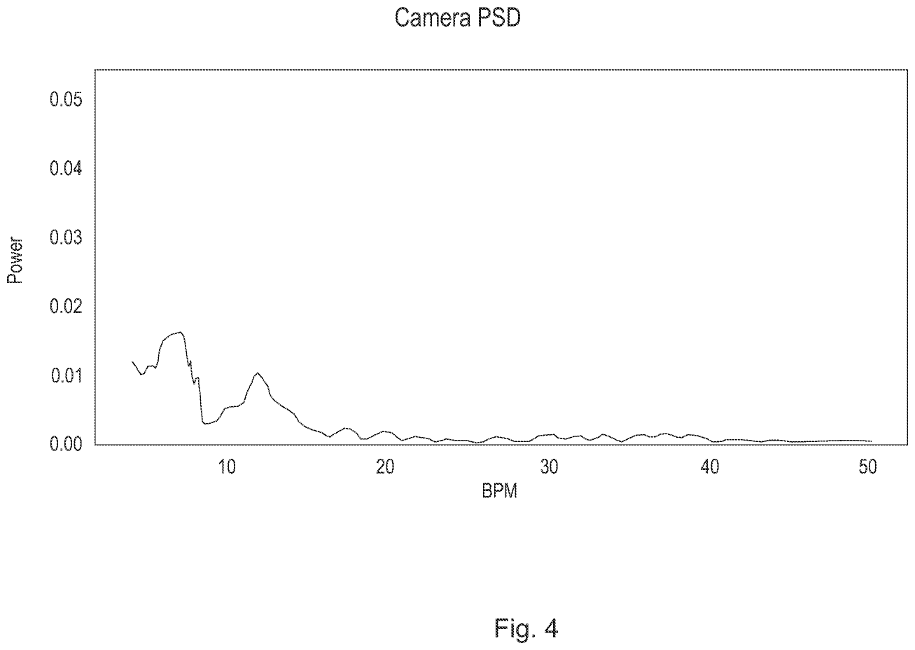

Further to the example of FIG. 2A, the chest movement signal 302 is provided to displacement power spectrum density (PSD) estimator 115. The PSD analysis of the displacement PSD estimator 115 may involve Fourier analysis or Lomb-Scargle analysis. The displacement PSD estimator 115 generates a displacement PSD signal 114. An example of the displacement PSD signal 114 derived based on a chest movement signal 302 captured by a camera is shown in FIG. 4.

The displacement PSD signal 114 is provided to displacement respiration rate estimator 117 to determine an estimated displacement respiration rate 104. In some examples, the estimated displacement respiration rate 104 is determined based on the value of the displacement PSD signal 114 over the respiration rate frequency range 116. The respiration rate frequency range 116 may correspond to a range of possible human respiration rates. Further, the displacement PSD signal 114 is also used to determine a displacement SQI 108. The SQI of the chest movement signal 302 is evaluated based on the breadth of the power spectrum. A good quality, high SQI signal will have a narrow spectrum (indicative of a strong signal above the noise floor), while a poor quality, low SQI signal with noise will have a broad and flatter spectrum. Like the audio SQI 106, the displacement SQI 108 is also determined according to the predefined SQI epoch length 112 (such as 30 seconds).

A data fuser 121 then determines and outputs a fused estimated respiration rate 110 based on the estimated audio respiration rate 102, the estimated displacement respiration rate 104, the audio SQI 106, and the displacement SQI 108. In determining the fused estimated respiration rate 110, the data fuser 121 may use weighted averaging to weigh respiration rate estimations based on high SQI signals more heavily than respiration rate estimations based on low SQI signals. For example, if the audio SQI 106 indicates a noisy, low quality, audio recording signal 202, and the displacement SQI 108 indicates a strong, high quality, chest movement signal 302, the estimated displacement respiration rate 106 will be weighted more heavily than the estimated audio respiration rate 102 in the fused estimated respiration rate 110. The fused estimated respiration rate 110 is then transmitted, via wired or wireless connection, to the display 400 to be shown on a display screen. In further examples, the fused estimated respiration rate 110 may be transmit, via wired or wireless connection, to an external infection monitoring system for further processing and analysis.

FIG. 2B shows a variation of the system 10 of FIG. 2A generating the fused estimated respiration rate 110 based on two chest movement signals 302a, 302b provided by two different types of displacement sensors 300a. In the non-limiting example of FIG. 2B, the first displacement sensor 300a may be a camera, such as a Sony DSCR10 camera or a Nikon Coolpix P11 camera. Further, the second displacement sensor 300b may be a laser rangefinder, such as a Leica D810 rangefinder. As described with reference to FIG. 2A, the processor 125 determines a first displacement respiration rate signal 104a and a first displacement SQI 108a based on the first chest movement signal 302a (corresponding to a camera). In some examples, the first chest movement signal 302a is generated through processing, via a video processor 123, video data 301a captured by the camera into a one-dimensional signal representative of chest movement. The processor 125 also determines a second displacement respiration rate signal 104b and a second displacement SQI signal 108b based on the second chest movement signal 302b (corresponding to a laser rangefinder). Unlike the data provided by the camera, distance data provided by the laser rangefinder is already a one-dimensional signal representative of chest movement. The data fuser 121 then fuses, using weighted averaging, the first and second displacement signals 104a, 104b based on the first and second displacement SQI signals 108a, 108b to generate the fused estimated respiration rate 110.

FIG. 2C shows a variation of the systems 10 of FIGS. 2A and 2B. In particular, the system 10 of FIG. 2C incorporates all three sensor types (audio sensor 200, first displacement sensor 300a, and second displacement sensor 300b) described above. Thus, the data fuser 121 determines, via weighted averaging, the fused estimated respiration rate 110 by combining the estimated audio respiration rate 102 (based on data captured by, for example, a parabolic microphone), the first estimated displacement respiration rate 104a (based on data captured by, for example, a camera), and the second estimated displacement respiration rate 104b (based on data captured by, for example, a laser rangefinder) based on signal quality indexes 106, 108a, 108b associated with each sensor 200, 300a, 300b.

FIG. 5 schematically illustrates the controller 100 previously depicted in FIG. 1. The controller 100 includes the processor 125, the memory 175, and the transceiver 185. The processor 125 is configured to execute the audio respiration rate estimator 111, the audio SQI estimator 113, the displacement PSD estimator 115, the displacement respiration rate estimator 117, the display SQI estimator 119, the data fuser 121, and the video processor 123. The memory 175 is configured to store the audio recording signal 202 provided by the audio sensor 200, the chest movement signal 302 provided by the displacement sensor 300, the estimated audio respiration rate 102, the estimated displacement respiration rate 104, the audio SQI 106, the displacement SQI 108, the fused estimated respiration rate 110, the SQI epoch length 112, the displacement PSD signal 114, the audio respiration rate frequency rate 116, and the audio total frequency range 118.

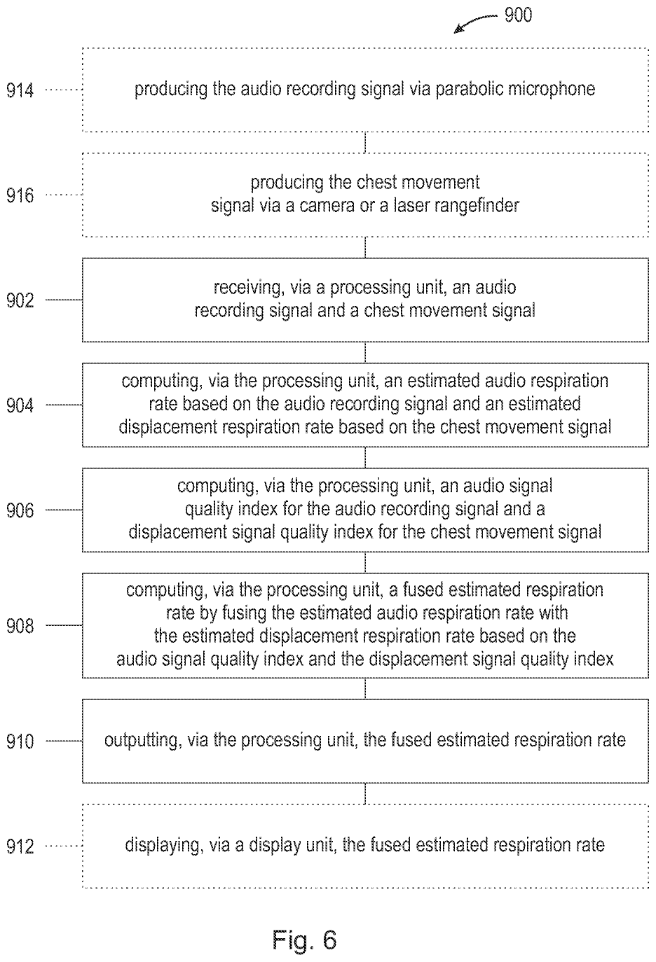

FIG. 6 is a flow chart of a method 900 for estimating respiration rate. Referring to FIGS. 1-6, the method 900 includes, in step 902, receiving, via a processing unit 125, an audio recording signal 202 and a chest movement signal 302.

The method 900 further includes, in step 904, computing, via the processing unit 125, an estimated audio respiration rate 102 based on the audio recording signal 202 and an estimated displacement respiration rate 104 based on the chest movement signal 302.

The method 900 further includes, in step 906, computing, via the processing unit 125, an audio signal quality index 106 for the audio recording signal 102 and a displacement signal quality index 108 for the chest movement signal 104.

The method 900 further includes, in step 908, computing, via the processing unit 125, a fused estimated respiration rate 110 by fusing the estimated audio respiration rate 102 with the estimated displacement respiration rate 104 based on the audio signal quality index 106 and the displacement signal quality index 108.

The method 900 further includes, in step 910, outputting, via the processing unit 125, the fused estimated respiration rate 110.

According to an example, the method 900 further includes, in optional step 912, displaying, via a display unit 400, the fused estimated respiration rate 110.

According to an example, the method 900 further includes, in optional step 914, producing the audio recording signal 202 via parabolic microphone.

According to an example, the method 900 further includes producing the chest movement signal via a camera or a laser rangefinder.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects may be implemented using hardware, software, or a combination thereof. When any aspect is implemented at least in part in software, the software code can be executed on any suitable processor or collection of processors, whether provided in a single device or computer or distributed among multiple devices/computers.

The present disclosure may be implemented as a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions may be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant may be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific examples described herein. It is, therefore, to be understood that the foregoing examples are presented by way of example only and that, within the scope of the appended claims and equivalents thereto, examples may be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A system for estimating respiration rate, the system comprising:
an audio sensor configured to produce an audio recording signal;
a displacement sensor configured to produce a chest movement signal; and
a processing unit configured to:
receive the audio recording signal and the chest movement signal;
compute an estimated audio respiration rate based on the audio recording signal and an estimated displacement respiration rate based on the chest movement signal;
compute an audio signal quality index for the audio recording signal and a displacement signal quality index for the chest movement signal;
compute a fused estimated respiration rate by fusing the estimated audio respiration rate with the estimated displacement respiration rate based on the audio signal quality index and the displacement signal quality index; and
output the fused estimated respiration rate.

2. The system of claim 1 wherein the audio signal quality index and/or the displacement signal quality index is computed in epochs of 30 seconds.

3. The system of claim 1, wherein the fusing of the estimated audio respiration rate and the estimated displacement respiration rate is done by using weighted averaging of the audio signal quality index and the displacement signal quality index.

4. The system of claim 1, wherein the estimated audio respiration rate is determined through spectrogram analysis of the audio recording signal.

5. The system of claim 1, wherein the estimated displacement respiration rate is calculated based on a power spectrum density of the chest movement signal.

6. The system of claim 1, wherein the audio signal quality index is calculated based on a respiration rate frequency range and a total frequency range of the audio recording signal.

7. The system of claim 1, wherein the displacement signal quality index is calculated based on a power spectrum density of the chest movement signal.

8. The system of claim 1, further comprising a display unit configured to display the fused estimated respiration rate.

9. The system of claim 1, wherein the audio sensor is a parabolic microphone.

10. The system of claim 1, wherein the displacement sensor is a camera.

11. The system of claim 1, wherein the displacement sensor is a laser rangefinder.

12. A method for estimating respiration rate, comprising:
receiving, via a processing unit, an audio recording signal and a chest movement signal;
computing, via the processing unit, an estimated audio respiration rate based on the audio recording signal and an estimated displacement respiration rate based on the chest movement signal;
computing, via the processing unit, an audio signal quality index for the audio recording signal and a displacement signal quality index for the chest movement signal;
computing, via the processing unit, a fused estimated respiration rate by fusing the estimated audio respiration rate with the estimated displacement respiration rate based on the audio signal quality index and the displacement signal quality index; and
outputting, via the processing unit, the fused estimated respiration rate.

13. The method of claim 12, further comprising displaying, via a display unit, the fused estimated respiration rate.

14. The method of claim 12, further comprising producing the audio recording signal via parabolic microphone.

15. The method of claim 12, further comprising producing the chest movement signal via a camera or a laser rangefinder.
